(19) United States
(12) Patent Application Publication
(10) Pub. No.: US 2026/0166188 A1
(43) Pub. Date: Jun. 18, 2026

(54) BIDET

(71) Applicant: COWAY Co., Ltd., Gongju-si (KR)

(72) Inventors: Dong Ik NAM, Seoul (KR); Namgyu LEE, Seoul (KR); Gyu Jae JEONG, Seoul (KR); Seong Eun LIM, Seoul (KR)

(73) Assignee: COWAY Co., Ltd., Gongju-si (KR)

(21) Appl. No.: 19/422,601

(22) Filed: Dec. 17, 2025

(30) Foreign Application Priority Data
Dec. 18, 2024 (KR) 10-2024-0190227
Dec. 24, 2024 (KR) 10-2024-0195973

(51) Int. Cl. A61L 2/104; E03D 9/08

(52) U.S. Cl. CPC A61L 2/104; E03D 9/08; A61L 2202/11; A61L 2202/14; A61L 2202/17

(57) ABSTRACT

A bidet includes: a bidet main body configured to be installed on a toilet bowl and providing a nozzle housing; a nozzle disposed within the nozzle housing, extending in one direction, and spraying cleansing water to a user; and a sterilization module that sterilizes the nozzle using ultraviolet light. The nozzle includes a nozzle body that provides a space in which the cleansing water flows; and a protrusion that prevents the ultraviolet light from being emitted through a gap between the nozzle housing and the nozzle body.

### TECHNICAL FIELD

The present disclosure relates to a bidet.

### BACKGROUND

A bidet is a device configured to automatically perform anus cleaning and female private part cleaning by spraying cleansing water through a nozzle. Furthermore, in a bidet, after cleaning using cleansing water is completed, the bidet may be operated to dry the cleaned area by spraying air from a drying device provided inside the bidet.

When a user uses a cleansing or bidet function, the cleansing water sprayed from the nozzle directly contacts the user's body. When contaminants remain on the nozzle or when microorganisms have propagated, it may cause diseases such as anal disorders to the user. Therefore, the nozzle of the bidet should always be maintained in a clean state.

In this regard, Korean Patent Application Publication No. 10-2024-0163931, titled “Sterilization Control Method for Bidet” by the applicant NCM Co., Ltd. (Patent Document 1), discloses a sterilization control system and method for a bidet in which sterilizing water is generated, and the generated sterilizing water sterilizes a cleansing flow path, a bidet flow path, the surface of a nozzle body, and the toilet bowl on which the bidet is installed.

However, according to Patent Document 1, since the generated sterilizing water sterilizes the cleansing flow path, the bidet flow path, the nozzle body, and even the toilet bowl, the sterilizing water is emitted to the outside of the bidet main body and comes into contact with the user's skin. Therefore, Patent Document 1 has a problem in that it must additionally perform a sterilizing water removal step of removing the sterilizing water to prevent such sterilizing water from contacting the user's skin.

Furthermore, Patent Document 1 has a problem in that water on the surface of the nozzle is not drained before sterilization. When the water is not drained before being cleaned, a problem may arise in that the overall sterilization effect may be reduced because the parts of the nozzle where water is present on the surface are not sterilized. In other words, there is a problem that only the dry parts without water on the nozzle surface are sterilized, while the parts with water are not sterilized, leaving the unsterilized parts still unhygienic.

Furthermore, Korean Patent Application Publication No. 10-2022-0056512, titled “Hot Water Cleaning Device Having Lighting Lamp” by the applicant Kohler Novita Co., Ltd. (Patent Document 2), discloses that a waterproof cover is disposed on the bidet main body in order to prevent water from flowing in toward a lighting lamp disposed inside the bidet main body. In other words, in Patent Document 2, a waterproof cover is separately disposed on the bidet main body for internal waterproofing of the bidet main body.

However, Patent Document 2 has a problem in that the waterproof cover merely prevents water from flowing in toward the lighting lamp, but it cannot prevent light leakage from occurring from the lighting lamp through a gap in the waterproof cover. Moreover, since the lighting lamp of Patent Document 2 is for providing illumination to the inside of the toilet bowl and not for sterilization, there is a problem that the function of the lighting lamp may not be achieved when it is configured to block the light from the lighting lamp. In other words, the lighting lamp merely provides light to the inside of the toilet bowl and does not have a separate function of sterilizing the nozzle. Therefore, Patent Document 2 does not recognize the problems that may arise when a bidet is configured to sterilize the nozzle using ultraviolet light.

Against this background, a need is emerging for a bidet that sterilizes the nozzle with ultraviolet light without cleansing water.

Furthermore, Korean Patent Publication No. 10-1772190, titled “Sterilization System for Bidet” by the applicants NCM Co., Ltd. and Cuckoo Holdings Co., Ltd. (Patent Document 3), discloses a bidet sterilization system for sterilizing a water tank, a cleaning flow path, a displacement flow path, and the surface of a nozzle body, and for removing residual sterilizing water after cleaning.

However, Patent Document 3 also has a problem in that water may remain on the surface of the nozzle before sterilization. The nozzle is provided to clean the user by spraying water, and a lot of water may remain on the nozzle surface after cleaning is provided. When the nozzle is sterilized immediately without the water on the nozzle surface being drained, there is a problem in that the parts of the nozzle surface where water is present are not sterilized, thereby reducing the overall sterilization effect.

### SUMMARY

Embodiments of the present disclosure, conceived against the background described above, aim to provide a bidet that sterilizes a nozzle and a nozzle housing using ultraviolet light.

Furthermore, embodiments of the present disclosure aim to provide a bidet in which the nozzle may be more effectively sterilized by being configured to prevent ultraviolet light from being emitted to the outside of the nozzle housing when a sterilization step is performed using ultraviolet light.

Furthermore, embodiments of the present disclosure aim to provide a bidet that prevents a user from being exposed to ultraviolet light by being configured to prevent ultraviolet light from being emitted to the outside of the nozzle housing when the sterilization step is performed.

Furthermore, embodiments of the present disclosure aim to provide a bidet that provides convenience to a user by allowing the sterilization step to be performed not only when the user operates a sterilization operation button but also at a cycle designated by the user.

Embodiments of the present disclosure, conceived against the background described above, aim to provide a bidet in which water around the nozzle is drained before the nozzle is sterilized.

In accordance with a first aspect of the present disclosure, there is provided a bidet, including: a bidet main body configured to be installed on a toilet bowl and providing a nozzle housing; a nozzle disposed within the nozzle housing, extending in one direction, and spraying cleansing water to a user; and a sterilization module that sterilizes the nozzle using ultraviolet light, wherein the nozzle includes a nozzle body that provides a space in which the cleansing water flows; and a protrusion that prevents the ultraviolet light from being emitted through a gap between the nozzle housing and the nozzle body.

Further, the sterilization module may be disposed on an upper side of the nozzle, the nozzle may include one or more spray holes that provide a passage through which cleansing water is sprayed to the user, and the sterilization module may irradiate the one or more spray holes with ultraviolet light.

Further, the protrusion may extend along a circumferential direction of the nozzle body, and the protrusion may be formed at an end of the nozzle body and has a tapered shape in which a diameter of an outer circumferential surface of the nozzle body increases toward a distal end in the one direction.

Further, the bidet may further include: a controller configured to control the nozzle. The controller may control the nozzle to perform a standby step before the user is seated, a user function step in which the nozzle protrudes to an outside of the bidet main body and sprays cleansing water to the user based on a user's operation, and a sterilization step in which the nozzle is sterilized by ultraviolet light, and, in the sterilization step, the controller may control movement of the nozzle such that the nozzle is inserted further inside the bidet main body than a position of the nozzle in the user function step.

Further, the sterilization module may be disposed to irradiate ultraviolet light at an angle inclined with respect to the one direction in which the nozzle body extends.

Further, the nozzle housing may include a corresponding part that is formed to correspond to the shape of the protrusion and engages with the protrusion, so as to prevent ultraviolet light from being emitted to the outside of the nozzle housing when the sterilization step is performed.

Further, the nozzle body may further include: a cover part that provides a surface formed continuously with a front end of the nozzle housing when either the standby step or the sterilization step is performed; and a door that is selectively opened and closed to cover the cover part.

Further, the one or more spray holes may be disposed within a region that is irradiated with ultraviolet light from the sterilization module when viewed from the one direction.

Further, a nozzle packing part may be provided on an outer circumferential surface of the protrusion, which is elastically compressed during the sterilization step to seal a gap between the nozzle housing and the protrusion.

Further, the nozzle housing may be provided with a main body packing part that is disposed between the nozzle housing and the protrusion so as to be elastically compressed during the sterilization step.

In accordance with a second aspect of the present disclosure, there is provided a bidet including: a bidet main body configured to be installed on a toilet bowl; a nozzle provided in the bidet main body and spraying cleansing water to a user; and a controller configured to control operation of the nozzle, wherein the controller controls the nozzle to perform a standby step in which the nozzle is in a retracted state inside the bidet main body, a user function step in which the nozzle protrudes outside the bidet main body and sprays cleansing water to the user based on the user's operation, a sterilization preparation step in which a state where the nozzle protrudes outside the bidet main body is maintained to drain water from around the nozzle, and a sterilization step in which the nozzle is sterilized by ultraviolet (UV) light, and wherein, in the sterilization preparation step, a state in which the nozzle protrudes more outward than a position of the nozzle in the sterilization step is maintained for a predetermined time.

Further, the bidet may further include a nozzle housing that provides a space in which the nozzle is accommodated. The nozzle and the nozzle housing may be configured to prevent ultraviolet light from being emitted to the outside of the nozzle housing when the sterilization step is performed.

Further, in the sterilization preparation step, a state in which the nozzle protrudes more outward than a position of the nozzle in the standby step may be maintained for a predetermined time.

Further, the bidet may further include an operating unit that includes a plurality of operating buttons operated by the user to activate at least one of the bidet main body and the nozzle. The plurality of operating buttons may include: a cleansing operation button operated by the user to cause the user function step to be performed; and a sterilization operation button operated by a user to cause the sterilization step to be performed. The sterilization step may be performed when the sterilization operation button is operated by the user.

Further, the bidet may further include: a toilet seat rotatably mounted to the bidet main body and configured for a user to be seated on; and a seat sensor that detects whether the user is seated on the toilet seat. When the sterilization step is not performed for a preset sterilization standby time after a previous sterilization step was performed, the controller may perform the sterilization step.

Further, the controller may control the nozzle to further perform a cleansing preparation step in which the nozzle is cleaned when the seat sensor detects that a user is seated, and a cleaning step in which the nozzle is cleaned.

Further, an extent to which the nozzle protrudes outward in the sterilization preparation step may be smaller than an extent to which the nozzle protrudes outward in the user function step, and may be the same as an extent to which the nozzle protrudes outward in the cleansing preparation step.

Further, a position where the nozzle is disposed in the sterilization preparation step may be closer to a position where the nozzle is disposed in the standby step than to a position where the nozzle is disposed in the user function step.

Further, the sterilization standby time may be longer than a time for which the cleansing preparation step is performed, and a time for which the sterilization preparation step is performed may be shorter than the sterilization standby time.

Further, an amount of water on an outer surface of the nozzle after the sterilization preparation step is completed may be less than at least one of an amount of water on the outer surface of the nozzle after the cleansing preparation step is completed, an amount of water on the outer surface of the nozzle after the user function step is completed, and an amount of water on the outer surface of the nozzle after the cleaning step is completed.

According to embodiments of the present disclosure, a nozzle and a nozzle housing may be effectively sterilized through ultraviolet light.

Furthermore, according to the embodiments of the present disclosure, the nozzle may be more effectively sterilized because the bidet is configured to prevent ultraviolet light from being emitted to the outside of the nozzle housing when a sterilization step is performed.

Furthermore, according to the embodiments of the present disclosure, there is an effect that a user is prevented from being exposed to ultraviolet light because the bidet is configured to prevent ultraviolet light from being emitted to the outside of the nozzle housing when the sterilization step is performed.

Furthermore, according to the embodiments of the present disclosure, there is an effect of providing convenience to the user by allowing the sterilization step to be performed not only when the user operates a sterilization operation button, but also at a cycle designated by the user.

Furthermore, according to the embodiments of the present disclosure, a sterilization step can be performed effectively by draining water from around a nozzle in a sterilization preparation step.

Furthermore, according to the embodiments of the present disclosure, water may be efficiently drained as a state in which the nozzle protrudes outward persists for a predetermined time in the sterilization preparation step.

Furthermore, according to the embodiments of the present disclosure, the nozzle may be effectively sterilized because the nozzle and a nozzle housing are configured to prevent ultraviolet light from being emitted to the outside of the nozzle housing when the sterilization step is performed.

Furthermore, the embodiments of the present disclosure have the effect of providing convenience to a user by allowing the sterilization step to be performed when the user operates a sterilization operation button.

Furthermore, the embodiments of the present disclosure have the effect of maintaining the nozzle hygienically by allowing the sterilization step to be performed after a sterilization standby time has elapsed, even if the user does not operate the sterilization operation button.

Furthermore, the embodiments of the present disclosure provides hygienic cleaning to a user by performing a cleansing preparation step of cleaning the nozzle when a user's seating is detected by a seat sensor.

Furthermore, in the embodiments of the present disclosure, power is not wasted because the sterilization standby time is provided to be longer than the time for which the cleansing preparation step is performed or the time for which the sterilization preparation step is performed.

Furthermore, according to the embodiments of the present disclosure, the nozzle may be effectively sterilized in the sterilization step by ensuring that there is almost no water on the outer surface of the nozzle after the sterilization preparation step is completed.

### BRIEF DESCRIPTION OF THE DRAWINGS

FIG. 1 is a conceptual diagram illustrating a bidet according to a first embodiment of the present disclosure.

FIG. 2 is a perspective view of the bidet in a cover open state according to the first embodiment of the present disclosure.

FIG. 3 is a diagram illustrating a nozzle, a sterilization module connected to the nozzle, and a door when the bidet is in a sterilization step according to the first embodiment of the present disclosure.

FIG. 4 is a diagram illustrating the nozzle, the sterilization module connected to the nozzle, and the door when the bidet is in a user function step according to the first embodiment of the present disclosure.

FIG. 5 is a front view of the nozzle, a motor, and the sterilization module when the bidet is in the user function step according to the first embodiment of the present disclosure.

FIG. 6 is a front view of the nozzle, the motor, and the sterilization module when the bidet is in the sterilization step according to the first embodiment of the present disclosure.

FIG. 7 is a side view of the nozzle, the sterilization module connected to the nozzle, and the door when the bidet is in the sterilization step according to the first embodiment of the present disclosure.

FIG. 8 is a cross-sectional view taken along the line VIII-VIII of FIG. 7.

FIG. 9 is a perspective view illustrating the nozzle of the bidet according to the first embodiment of the present disclosure.

FIG. 10 is a perspective view illustrating a state in which a nozzle packing part is disposed on the nozzle of the bidet according to the first embodiment of the present disclosure.

FIG. 11 is a perspective view illustrating a state in which a main body packing part is disposed in a nozzle housing of a bidet according to a second embodiment of the present disclosure.

FIG. 12 is a flowchart showing a sequence in which a bidet is controlled according to a third embodiment of the present disclosure.

### DETAILED DESCRIPTION OF THE INVENTION

Hereinafter, a configuration and operation according to an embodiment of the present disclosure will be described in detail with reference to the accompanying drawings.

Further, in describing the present disclosure, detailed descriptions of known configurations or functions may be omitted to clarify the present disclosure.

When an element is referred to as being ‘connected’ to or ‘supplied’ to another element, it should be understood that the element may be directly connected to or supplied to the other element, but that other elements may exist in the middle.

The terms used in the present disclosure are only used for describing specific embodiments, and are not intended to limit the present disclosure. Singular expressions include plural expressions unless the context clearly indicates otherwise.

Further, in the present disclosure, it is to be noted that expressions, such as the upper side, the lower side, and the side surface, are described based on the illustration of drawings, but may be modified if directions of corresponding objects are changed. For the same reasons, some components are exaggerated, omitted, or schematically illustrated in the accompanying drawings, and the size of each component does not fully reflect the actual size.

Terms including ordinal numbers, such as first and second, may be used for describing various elements, but the corresponding elements are not limited by these terms. These terms are only used for the purpose of distinguishing one element from another element.

In the present specification, it is to be understood that the terms such as “including” are intended to indicate the existence of the certain features, areas, integers, steps, actions, elements, combinations, and/or groups thereof disclosed in the specification, and are not intended to preclude the possibility that one or more other certain features, areas, integers, steps, actions, elements, combinations, and/or groups thereof may exist or may be added.

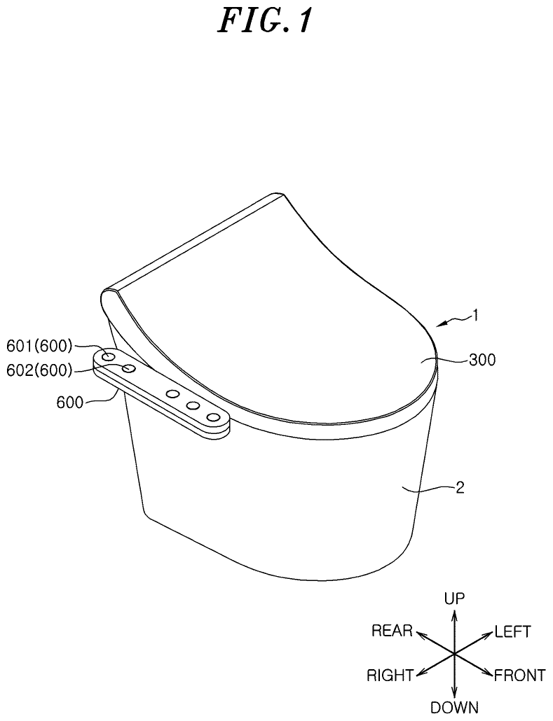

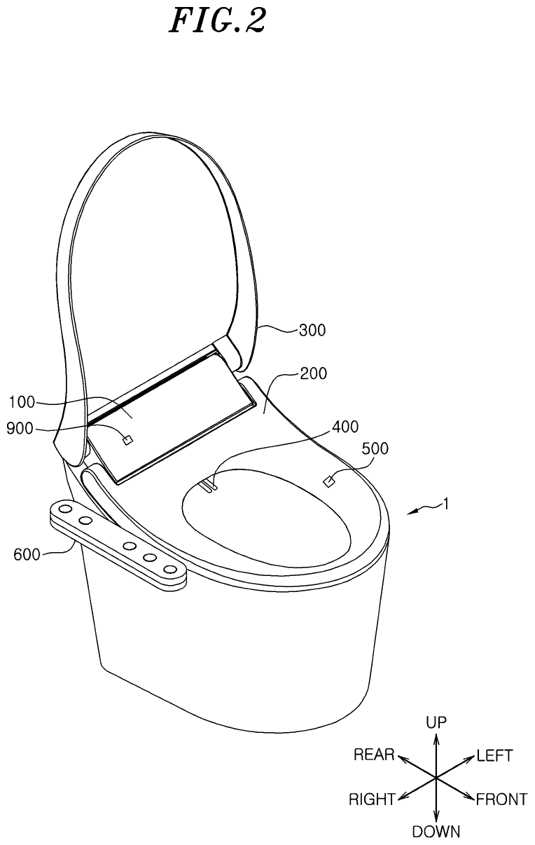

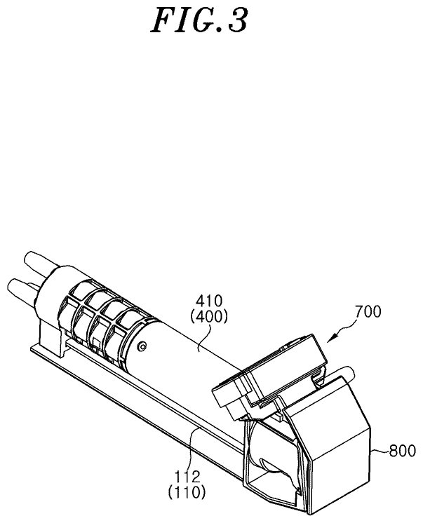

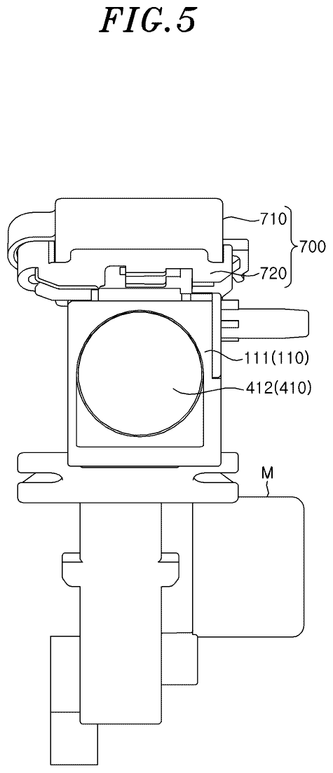

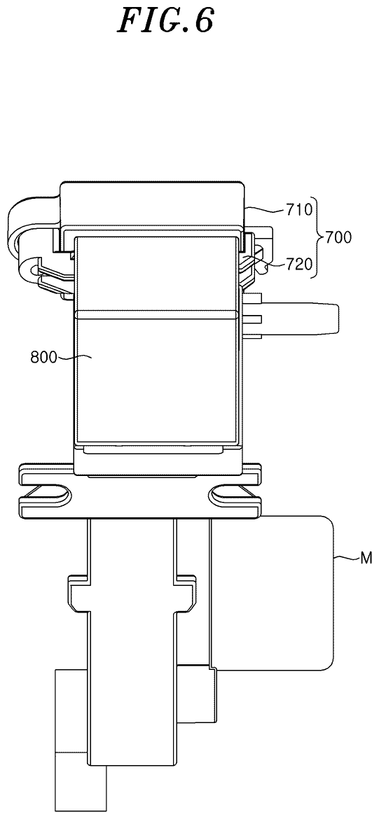

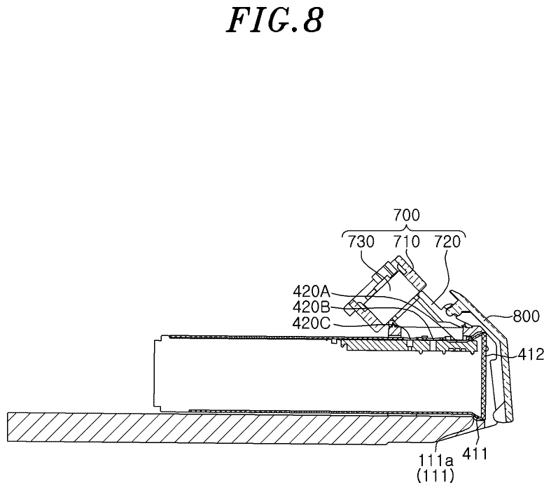

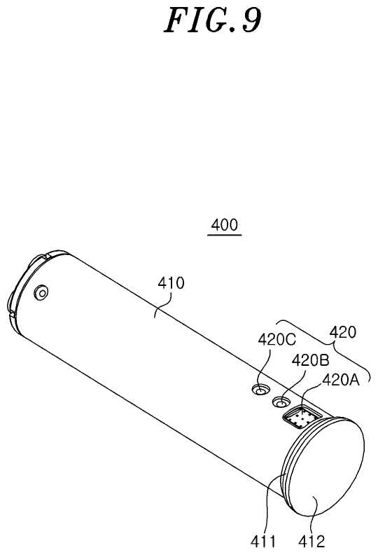

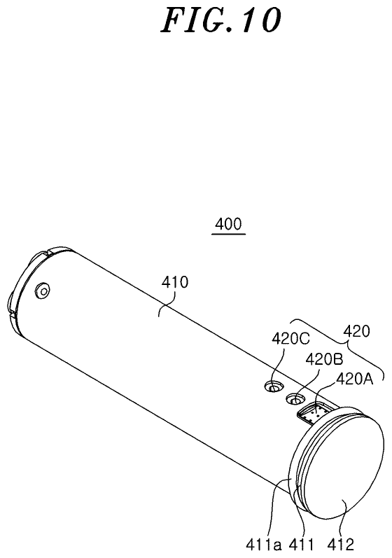

Hereinafter, a specific configuration of a bidet 1 according to a first embodiment of the present disclosure will be described with reference to FIGS. 1 to 10.

Referring to FIGS. 1 to 10, the bidet 1 according to the first embodiment of the present disclosure may clean a user's body by spraying cleansing water toward the user. The bidet 1 may be installed on a toilet bowl 2. Furthermore, the bidet 1 may include a bidet main body 100, a toilet seat 200, a cover 300, a nozzle 400, a motor M, an operating unit 600, a sterilization module 700, a door 800, and a controller 900.

The bidet main body 100 may be configured to be installed on the ceramic body of a toilet bowl, and may accommodate a container (not shown) for accommodating cleansing liquid, liquid, etc., the nozzle 400, a fan (not shown) for providing a drying function to the user, etc. The bidet main body 100 may support the cover 300 so as to be detachable. Furthermore, the bidet main body 100 may be installed on the toilet bowl 2. A nozzle housing 110 may be provided in the bidet main body 100.

The nozzle housing 110 may provide a space in which the nozzle 400 is accommodated. The nozzle housing 110 may be configured to prevent ultraviolet light from being emitted to the outside of the nozzle housing 110 in a sterilization step, which will be described later. Furthermore, the nozzle housing 110 may include a first nozzle housing 111 and a second nozzle housing 112.

The first nozzle housing 111 may accommodate one end of the nozzle 400 when the nozzle 400 is moved rearward and inserted into the nozzle housing 110. The first nozzle housing 111 may be disposed at a front side of the second nozzle housing 112. Furthermore, a circular cavity may be formed in the first nozzle housing 111 so that the nozzle 400 is accommodated therein. A corresponding part 111a may be formed in the first nozzle housing 111.

The corresponding part 111a may block ultraviolet light to prevent the ultraviolet light from being emitted to the outside of the nozzle housing 110 when the sterilization step, in which the spray hole 420 of the nozzle 400 is sterilized by the sterilization module 700, is performed. The corresponding part 111a may be formed to correspond to the shape of the protrusion 411. In other words, the corresponding part 111a may be formed to engage with the protrusion 411. For example, when the protrusion 411 has a tapered shape, the corresponding part 111a may also have a tapered shape.

The second nozzle housing 112 may support the nozzle body 410. The second nozzle housing 112 may extend in one direction. The second nozzle housing 112 may guide the nozzle to move in a front and rear direction between a position where the nozzle 400 is inserted into the nozzle housing 110, as in a standby step, and a position where it protrudes to spray cleansing water to a user, as in a user function step. Furthermore, the length of the second nozzle housing 112 in one direction may be longer than the length of the nozzle 400 in one direction. At least a part of the second nozzle housing 112 may be in contact with the motor M.

The toilet seat 200 may provide a space for a user to be seated on. The toilet seat 200 may be rotatably coupled to the bidet main body 100. In other words, the toilet seat 200 may be configured to be rotatable with respect to the bidet main body 100 between a seatable position that covers the toilet bowl 2 and a toilet-exposed position that does not cover the toilet bowl 2 and is raised with respect to the toilet bowl 2. A seating surface on which a user is seated may be formed on the toilet seat 200. The toilet seat 200 may be rotated between a seat-allowing state that allows a user to be seated and in which the seating surface faces upward, and a seat-disallowing state in which it is rotated rearward so that the user cannot be seated.

The cover 300 may be rotatably mounted to the bidet main body 100. The cover 300 may be configured to be rotatable with respect to the bidet main body 100 between a cover closed position that closes the inside of the toilet bowl 2 to the outside and a cover open position that opens the inside of the toilet bowl 2 to the outside. The cover 300 may not expose the toilet bowl 2 to the user in the cover closed position. The cover 300 may be disposed at a higher position than the toilet seat 200 when the cover is in the cover closed position while the toilet seat 200 is positioned in the seat-allowing state.

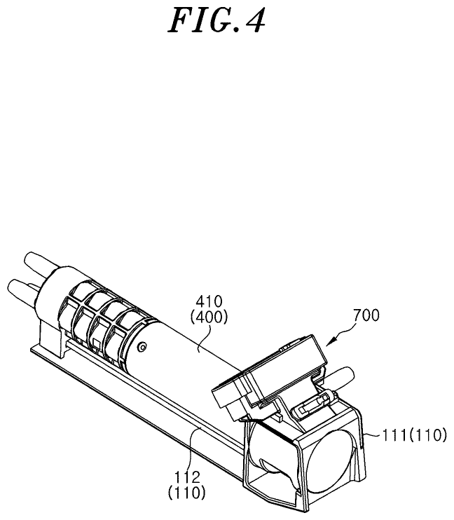

Referring further to FIGS. 4, 5, and 7-9, the nozzle 400 may discharge cleansing liquid, liquid, etc., toward the toilet bowl 2, or spray it toward the front where a user is located. The nozzle 400 may be provided in the bidet main body 100. Furthermore, the nozzle 400 may be disposed below the toilet seat 200. The nozzle 400 may be moved in the front and rear direction by the operation of the motor M. Furthermore, the nozzle 400 may extend in one direction. The nozzle 400 may include a nozzle body 410 and a spray hole 420.

The nozzle body 410 may provide a space in which cleansing water flows. The spray hole 420 may be formed in the nozzle body 410. Furthermore, the nozzle body 410 may have a shape that extends in one direction. The nozzle body 410 may be supported by the nozzle housing 110. A user-replaceable nozzle tip (not shown) may be formed at an end of the nozzle body 410. Furthermore, the nozzle body 410 may include a protrusion 411 and a cover part 412.

The nozzle body 410 may be disposed in the bidet main body 100. The nozzle body 410 may include a spray hole 420 for spraying water to a user and a cleaning water hole (not shown) for spraying water into the toilet bowl (2). Furthermore, the nozzle body 410 may be configured such that at least one of the water sprayed into the toilet bowl (2) and the drained water is discharged through the spray hole 420. The nozzle body 410 has a shape extending in one direction and may be accommodated inside the nozzle housing 110. The nozzle body 410 may be configured to prevent ultraviolet light from being emitted to the outside of the nozzle housing 110 in a sterilization step S600, which will be described later.

The protrusion 411 may prevent ultraviolet light from being emitted through a gap between the nozzle housing 110 and the nozzle 400. The protrusion 411 may extend along a circumferential direction of the nozzle body 410, and may have a shape that protrudes in a direction deviating from the direction in which the nozzle body 410 extends. Furthermore, the protrusion 411 may be formed at an end of the nozzle body 410. Furthermore, the protrusion 411 may also be formed on a user-replaceable nozzle tip when the nozzle tip is formed at the end of the nozzle body 410. The protrusion 411 may have a tapered shape in which a diameter of an outer circumferential surface of the nozzle body 410 increases toward a distal end in the one direction. A nozzle packing part 411a may be formed on the protrusion 411.

The nozzle packing part 411a may seal a gap between the nozzle housing 110 and the protrusion 411 by being compressed during the sterilization step. The nozzle packing part 411a may be formed of a material having elasticity. For example, the nozzle packing part 411a may be a rubber O-ring.

The cover part 412 may cover a front end of the nozzle housing 110 when either the standby step or the sterilization step is performed. The cover part 412, when covering the front end of the nozzle housing 110, may provide a surface formed continuously with the front end of the nozzle housing 110. The cover part 412 may be rotated about a rotation axis to selectively cover the front end of the nozzle housing 110.

The spray hole 420 may provide a passage through which cleansing water is sprayed to the user. One or more spray holes 420 may be provided. Furthermore, the spray hole 420 may be disposed within a region that is irradiated with ultraviolet light from the sterilization module 700 when viewed from one direction. The spray hole 420 may include a first spray hole 420A, a second spray hole 420B, and a third spray hole 420C.

The first spray hole 420A may be disposed at the forefront among the plurality of spray holes 420. In other words, the first spray hole 420A may be disposed at the outermost end of the nozzle body 410 in one direction. The cleansing water sprayed from the first spray hole 420A may be used for female hygiene. Furthermore, the pressure of the cleansing water sprayed from the first spray hole 420A may be smaller than the pressure of the cleansing water sprayed from the second spray hole 420B and the third spray hole 420C. The first spray hole 420A may be sterilized by ultraviolet light irradiated from the sterilization unit 730.

The second spray hole 420B may be disposed between the first spray hole 420A and the third spray hole 420C in one direction. The second spray hole 420B may be disposed on an upper side of the nozzle body 410. Furthermore, the second spray hole 420B may be sterilized by ultraviolet light irradiated from the sterilization unit 730.

The third spray hole 420C may be disposed at the rearmost among the plurality of spray holes 420. In other words, the third spray hole 420C may be disposed at the innermost side of the nozzle body 410 in one direction when the nozzle 400 is moved rearward and inserted into the nozzle housing 110. Furthermore, the third spray hole 420C may be formed on the upper side of the nozzle body 410. The third spray hole 420C may be sterilized by ultraviolet light irradiated from the sterilization unit 730.

The motor M may provide a driving force to the nozzle body 410 so that the nozzle body 410 is moved. When the nozzle body 410 is configured to be extendable, the nozzle body 410 may protrude outward or retract inward to be stored inside the bidet main body 100 by the driving force of the motor M. When the nozzle body 410 is configured to be slidable, the nozzle body 410 may be moved forward or rearward by the driving force of the motor M. The motor M may be in contact with the nozzle body 410.

The operating unit 600 may include a plurality of operating buttons pressed by a user so that at least one of the bidet main body 100 and the nozzle 400 is operated. For example, through the operating unit 600, the user may adjust the spray pressure of the cleansing water sprayed from the nozzle 400, and the operating unit 600 may receive an input from the user as to whether to operate the cleaning function, drying function, sterilization function, and temperature control function of the bidet main body 100. The operating unit 600 may include an operation panel provided with a plurality of buttons for operation and an operating unit housing in which a power supply unit is installed. Furthermore, the operating unit 600 may be disposed on the left or right side of the bidet main body 100, or disposed on the left or right side of the upper part of the toilet bowl 2 and supported by the bidet main body 100.

The operating unit 600 may be operated by the user to control the nozzle 400. The operating unit 600 may be coupled to the bidet main body 100 or the toilet seat 200. The operating unit 600 may receive an input from the user for the spray pressure of the cleansing water sprayed from the nozzle 400. The operating unit 600 may be provided in the form of a remote control and may be disposed separately from the toilet bowl. The operating unit 600 may include a plurality of operating buttons operated by the user to activate at least one of the bidet main body 100 and the nozzle 400. The plurality of operating buttons may include a cleansing operation button 601 and a sterilization operation button 602.

The cleansing operation button 601 may be operated by the user to cause a user function step S300, which will be described later, to be performed. When the cleansing operation button 601 is operated by the user, the user function step S300 may be performed. One cleansing operation button 601 may be provided, or a plurality of them may be provided.

The sterilization operation button 602 may be operated by the user to cause a sterilization step S600 to be performed. When the sterilization operation button 602 is operated by the user, the sterilization step S600 may be performed. The user may operate the sterilization operation button 602 when it is determined that sterilization of the nozzle 400 is necessary. The user may operate the sterilization operation button 602 to sterilize the nozzle 400 and then operate the cleansing operation button 601 to cause the nozzle 400 to perform the user function step S300, or after the user function step S300 is performed, the user may operate the sterilization operation button 602 to sterilize the nozzle 400.

The seat sensor 500 may be disposed on the toilet seat 200 to detect whether a user is seated. The seat sensor 500 may include a pressure sensor. When a user sits on the toilet seat 200, the seat sensor 500 may detect the pressure from the user's body. The seat sensor 500 may recognize that the user is seated on the toilet seat 200 when a pressure equal to or greater than a preset value is measured.

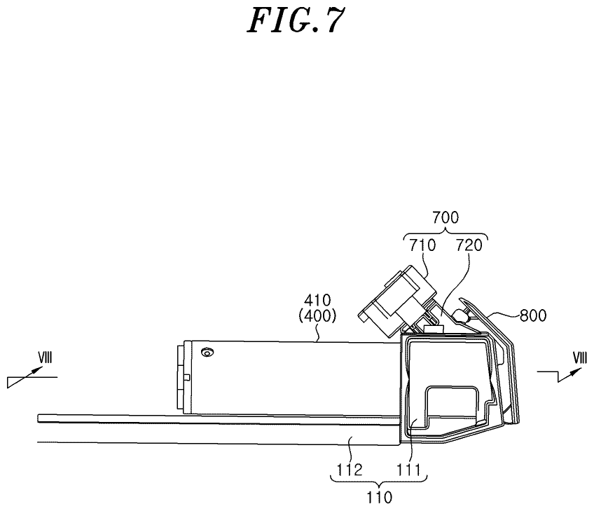

Referring further to FIGS. 7 to 10, the sterilization module 700 may sterilize the nozzle 400 using ultraviolet (UV) light. The sterilization module 700 may irradiate one or more spray holes 420 with ultraviolet light. Furthermore, the sterilization module 700 may be disposed on an upper side of the nozzle 400. The sterilization module 700 may be disposed to irradiate ultraviolet light at an angle inclined with respect to one direction in which the nozzle body 410 extends. The sterilization module 700 may be connected to a flow path that sprays cleaning water onto the surface of the spray hole 420. The cleaning water may include at least one of electrolyzed sterilizing water, bubble-cleansing sterilizing water, and plain water. Furthermore, the sterilization module 700 may include a support body 710, a connection body 720, and a sterilization unit 730.

The support body 710 may support the sterilization unit 730. In other words, the support body 710 may provide a space in which the sterilization unit 730 is accommodated. Furthermore, the support body 710 may be coupled to the connection body 720. The support body 710 may be formed at an angle inclined with respect to one direction in which the nozzle body 410 extends. For example, the support body 710 may be disposed inclined upward with respect to the nozzle body 410.

The connection body 720 may couple the sterilization unit 730 to the nozzle body 410. One side of the connection body 720 may be coupled to the nozzle body 410, and the other side, which is opposite to the one side, may be coupled to the support body 710. Furthermore, the connection body 720 may be formed at an angle inclined with respect to one direction in which the nozzle body 410 extends. However, the present embodiments are not limited thereto, and the connection body 720 may be formed integrally with the support body 710.

The sterilization unit 730 may sterilize the nozzle 400. The sterilization unit 730 may include an ultraviolet LED lamp. Furthermore, the ultraviolet light irradiated from the sterilization unit 730 may be UVC (ultraviolet C), and the UVC may have a wavelength of 200 nm to 280 nm. The sterilization unit 730 may be disposed inside the support body 710.

The door 800 may be selectively opened and closed to cover the cover part 412 when either the standby step or the sterilization step is performed. The door 800 may cover one end of the nozzle 400. In other words, when the nozzle 400 is not in use, the door 800 may cover one end of the nozzle 400, so that the nozzle 400 is not exposed to the outside. Furthermore, the door 800 may be rotatably supported by the bidet main body 100 or the sterilization module 700.

The controller 900 may control the nozzle 400 based on a user's operation. In one embodiment, the controller 900 may control the nozzle 400 to perform a standby step in which the user is not seated, a user function step in which the nozzle 400 protrudes to the outside of the bidet main body 100 and sprays cleansing water to the user based on the user's operation, and a sterilization step in which the nozzle 400 is sterilized by ultraviolet light. The controller 900 may control the movement of the nozzle 400 so that, in the sterilization step, the nozzle 400 is inserted further inside the bidet main body 100 than the position of the nozzle 400 in the user function step. The controller 900 may be implemented by a computing device including a microprocessor, and its implementation method is obvious to those skilled in the art, so further detailed description will be omitted.

Hereinafter, the operation and effects of the bidet 1 having the configuration as described above will be described.

The bidet 1 according to the first embodiment of the present disclosure may sterilize the nozzle 400 with ultraviolet light. The nozzle 400 may be sterilized when it is inserted inside the bidet main body 100. In other words, the nozzle 400 may be sterilized during a sterilization step in which it is inserted inside the bidet main body 100 and controlled to emit ultraviolet light. Furthermore, the nozzle 400 may not be sterilized during a user function step of spraying cleansing water to the user.

Furthermore, because the sterilization module 700 that emits ultraviolet light to sterilize the nozzle 400 is disposed inside the bidet main body 100 and a protrusion is formed, it is possible to prevent or minimize light leakage, where ultraviolet light is emitted to the outside through a gap between the nozzle 400 and the bidet main body 100. Even if ultraviolet light is emitted from the sterilization module 700 inside the bidet main body 100, the protrusion 411 of the nozzle 400 may be formed to extend along the circumferential direction of the nozzle body 410 to more effectively prevent light leakage of ultraviolet light between the nozzle housing 110 and the nozzle 400. Furthermore, the corresponding part 111a may be formed to correspond to the shape of the protrusion 411 when the nozzle body 410 is disposed inside the bidet main body 100 and the sterilization step is performed, thereby preventing ultraviolet light from being emitted to the outside of the nozzle housing 110.

Furthermore, because the door 800 is selectively opened and closed to cover the cover part 412 of the nozzle 400, when the nozzle 400 is in the standby step or the sterilization step, it may effectively prevent the ultraviolet light emitted from the sterilization module 700 from being emitted to the outside by covering one end of the nozzle 400.

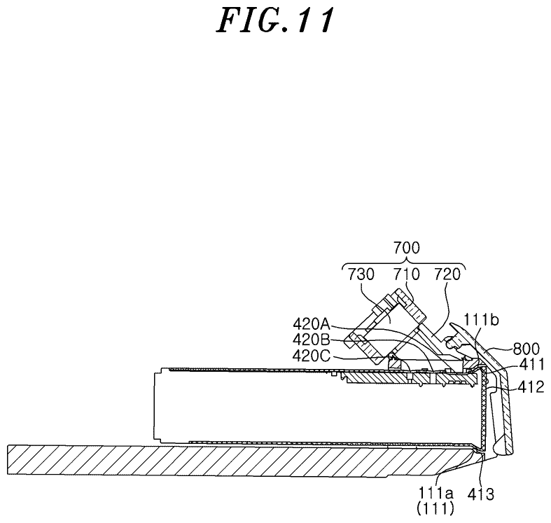

Meanwhile, in addition to this configuration, a bidet 1 according to a second embodiment of the present disclosure may be provided. Hereinafter, the second embodiment of the present disclosure will be described with reference to FIG. 11. In describing the second embodiment, the description will focus on the differences from the above-described embodiment, and for the same descriptions and reference numerals, reference is made to the above-described embodiment.

Referring to FIG. 11, a main body packing part 111b may be provided in the nozzle housing 110 of the bidet 1 according to the second embodiment of the present disclosure.

The main body packing part 111b may seal a gap between the nozzle housing 110 and the protrusion 411 by being compressed during the sterilization step. The main body packing part 111b may be formed to be elastically compressed. Furthermore, the main body packing part 111b may be disposed between the nozzle housing 110 and the protrusion 411. For example, the main body packing part 111b may be a rubber O-ring.

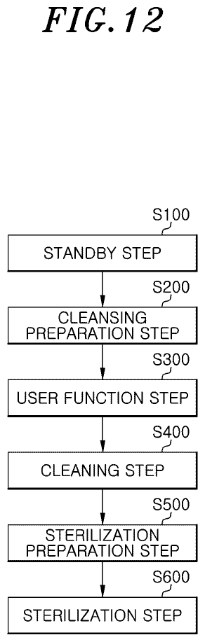

Meanwhile, in addition to this configuration, a bidet 1 according to a third embodiment of the present disclosure may be provided. Hereinafter, the third embodiment of the present disclosure will be described with reference to FIG. 12. In describing the third embodiment, the description will focus on the differences from the above-described embodiments, and for the same descriptions and reference numerals, reference is made to the above-described embodiments.

Hereinafter, the steps in which the controller 900 controls the nozzle 400 will be described with reference to FIG. 12. The controller 900 may control the nozzle 400 to perform a standby step S100 in which the nozzle 400 is in a retracted state inside the bidet main body 100, a cleansing preparation step S200 in which the nozzle 400 is cleaned with water or sterilizing water before a user function step S300, a user function step S300 in which the nozzle 400 protrudes to the outside of the bidet main body 100 and sprays cleansing water to the user based on the user's operation, a cleaning step S400 in which the nozzle 400 is cleaned with water or sterilizing water after the user function step S300, a sterilization preparation step S500 in which a state where the nozzle 400 protrudes outside the bidet main body 100 is maintained to drain water from around the nozzle 400, and a sterilization step S600 in which the nozzle 400 is sterilized by ultraviolet (UV) light.

In the standby step S100, the nozzle 400 may be stored inside the bidet main body 100. In the standby step S100, the user may not be seated on the toilet seat 200. In the standby step S100, one end of the nozzle 400 may be covered by the door 800.

In the cleansing preparation step S200, the nozzle 400 may be cleaned with water or sterilizing water. The sterilizing water may include electrolyzed water or sterilizing water including a bubble cleansing agent. In the cleansing preparation step S200, when a user's seating is detected by the seat sensor 500, the nozzle 400 may be cleaned with water or sterilizing water.

In the user function step S300, the nozzle 400 may spray water to the user according to the user's operation. In the user function step S300, cleansing may be provided to the user according to the cleansing operation button 601 operated by the user.

In the cleaning step S400, the nozzle 400 may be cleaned with water or sterilizing water. In the cleaning step S400, the nozzle 400 may be cleaned after providing cleaning to the user. The water on the outer surface of the nozzle 400 in the cleaning step S400 may be drained in the sterilization preparation step S500.

In the sterilization preparation step S500, water on the outer surface of the nozzle 400 may be drained. The water drained in the sterilization preparation step S500 may be the water used for cleaning the nozzle 400. In the sterilization preparation step S500, the nozzle 400 may protrude to the outside of the nozzle housing 110. In the sterilization preparation step S500, the nozzle 400 may protrude further outward by a predetermined preparation moving distance than the position of the nozzle 400 in the sterilization step S600. In the sterilization preparation step S500, the nozzle 400 may protrude further outward than the position of the nozzle 400 in the standby step S100. The extent to which the nozzle 400 protrudes outward in the sterilization preparation step S500 may be smaller than the extent to which the nozzle 400 protrudes outward in the user function step S300. In other words, the extent to which the nozzle 400 protrudes outward in the sterilization preparation step S500 may be smaller than the extent to which the nozzle 400 protrudes outward to provide cleaning to the user. The extent to which the nozzle 400 protrudes outward in the sterilization preparation step S500 may be the same as the extent to which the nozzle 400 protrudes outward in the cleansing preparation step S200.

The preparation moving distance in the sterilization preparation step S500 may be 1 mm to 3 mm. For example, the preparation moving distance in the sterilization preparation step S500 may be 2 mm. The position where the nozzle 400 is disposed in the sterilization preparation step S500 may be closer to the position where the nozzle 400 is disposed in the standby step S100 than the position where the nozzle 400 is disposed in the user function step S300.

The time for which the sterilization preparation step S500 is performed may be shorter than a sterilization standby time, which will be described later. The amount of water on the outer surface of the nozzle 400 after the sterilization preparation step S500 is completed may be less than at least one of the amount of water on the outer surface of the nozzle 400 after the cleansing preparation step S200 is completed, the amount of water on the outer surface of the nozzle 400 after the user function step S300 is completed, and the amount of water on the outer surface of the nozzle 400 after the cleaning step S400 is completed. There may be no water on the outer surface of the nozzle 400 after the sterilization preparation step S500 is completed. When there is no water on the outer surface of the nozzle 400 after the sterilization preparation step S500 is completed, the nozzle 400 may be effectively sterilized in the sterilization step S600.

In the sterilization step S600, the nozzle 400 may be sterilized by ultraviolet light. In the sterilization step S600, the nozzle 400 may be accommodated inside the nozzle housing 110 and sterilized by ultraviolet light. In the sterilization step S600, one end of the nozzle 400 may be covered by the door 800. The sterilization step S600 may be performed when the user operates the sterilization operation button 602 or when the sterilization step S600 is not performed for a sterilization standby time after the sterilization step S600 was performed. In the sterilization step S600, the nozzle 400 is sterilized by ultraviolet light, so the nozzle 400 may be maintained in a hygienic state. The time for which the sterilization step S600 is performed may be shorter than the standby step time for which the standby step S100 is performed, and longer than the time for which the user function step S300 is performed. The time for which the sterilization step S600 is performed may be 3 minutes to 5 minutes. The sterilization step S600 may be performed after the user function step S300 is performed.

When the sterilization step S600 is not performed for the sterilization standby time after the sterilization step S600 was performed, the controller 900 may perform the sterilization step S600. The sterilization standby time may be longer than the time for which the cleansing preparation step S200 is performed. The sterilization standby time may be 30 hours to 40 hours. For example, the sterilization standby time may be 36 hours.

Hereinafter, the effects of the bidet 1 having the configuration as described above will be described.

In the sterilization preparation step S500, the water around the nozzle 400 is drained, so that the sterilization step S600 may be effectively performed.

In the sterilization preparation step S500, the state in which the nozzle 400 protrudes outward persists for a predetermined time, so that water may be efficiently drained.

The nozzle 400 and the nozzle housing 110 are configured to prevent ultraviolet light from being emitted to the outside of the nozzle housing 110 when the sterilization step S600 is performed, so that the nozzle 400 may be effectively sterilized.

When the user operates the sterilization operation button, the sterilization step S600 is performed, thereby providing convenience to the user. Furthermore, even if the user does not operate the sterilization operation button, the sterilization step S600 is performed after the sterilization standby time has elapsed, so that the nozzle 400 may be maintained hygienically.

When a user's seating is detected by the seat sensor 500, the cleansing preparation step S200 in which the nozzle 400 is cleaned is performed, so that hygienic cleaning may be provided to the user.

The sterilization standby time is provided to be longer than the time for which the cleansing preparation step S200 is performed or the time for which the sterilization preparation step S500 is performed, so that power may not be wasted.

By ensuring that there is almost no water on the outer surface of the nozzle 400 after the sterilization preparation step S500 is completed, the nozzle may be effectively sterilized in the sterilization step S600.

The examples of the present disclosure have been described above as specific embodiments, but these are only examples, and the present disclosure is not limited thereto, and should be construed as having the widest scope according to the technical spirit disclosed in the present specification. A person skilled in the art may combine/substitute the disclosed embodiments to implement a pattern of a shape that is not disclosed, but it also does not depart from the scope of the present disclosure. In addition, those skilled in the art can easily change or modify the disclosed embodiments based on the present specification, and it is clear that such changes or modifications also belong to the scope of the present disclosure.

What is claimed is:

1. A bidet, comprising:
a bidet main body configured to be installed on a toilet bowl and providing a nozzle housing;
a nozzle disposed within the nozzle housing, extending in one direction, and spraying cleansing water to a user; and
a sterilization module that sterilizes the nozzle using ultraviolet light,
wherein the nozzle includes a nozzle body that provides a space in which the cleansing water flows; and
a protrusion that prevents the ultraviolet light from being emitted through a gap between the nozzle housing and the nozzle body.

2. The bidet of claim 1, wherein
the sterilization module is disposed on an upper side of the nozzle,
the nozzle comprises one or more spray holes that provide a passage through which cleansing water is sprayed to the user, and
the sterilization module irradiates the one or more spray holes with ultraviolet light.

3. The bidet of claim 1, wherein
the protrusion extends along a circumferential direction of the nozzle body, and
wherein the protrusion is formed at an end of the nozzle body and has a tapered shape in which a diameter of an outer circumferential surface of the nozzle body increases toward a distal end in the one direction.

4. The bidet of claim 3, further comprising:
a controller configured to control the nozzle,
wherein the controller controls the nozzle to perform a standby step before the user is seated, a user function step in which the nozzle protrudes to an outside of the bidet main body and sprays cleansing water to the user based on a user's operation, and a sterilization step in which the nozzle is sterilized by ultraviolet light, and
wherein, in the sterilization step, the controller controls movement of the nozzle such that the nozzle is inserted further inside the bidet main body than a position of the nozzle in the user function step.

5. The bidet of claim 3, wherein the sterilization module is disposed to irradiate ultraviolet light at an angle inclined with respect to the one direction in which the nozzle body extends.

6. The bidet of claim 4, wherein the nozzle housing includes a corresponding part that is formed to correspond to the shape of the protrusion and engages with the protrusion, so as to prevent ultraviolet light from being emitted to the outside of the nozzle housing when the sterilization step is performed.

7. The bidet of claim 4, wherein the nozzle body further includes:
a cover part that provides a surface formed continuously with a front end of the nozzle housing when either the standby step or the sterilization step is performed; and
a door that is selectively opened and closed to cover the cover part.

8. The bidet of claim 2, wherein the one or more spray holes are disposed within a region that is irradiated with ultraviolet light from the sterilization module when viewed from the one direction.

9. The bidet of claim 4, wherein a nozzle packing part is provided on an outer circumferential surface of the protrusion, which is elastically compressed during the sterilization step to seal a gap between the nozzle housing and the protrusion.

10. The bidet of claim 4, wherein the nozzle housing is provided with a main body packing part that is disposed between the nozzle housing and the protrusion so as to be elastically compressed during the sterilization step.

11. A bidet, comprising:
a bidet main body configured to be installed on a toilet bowl;
a nozzle provided in the bidet main body and spraying cleansing water to a user; and
a controller configured to control operation of the nozzle,
wherein the controller controls the nozzle to perform a standby step in which the nozzle is in a retracted state inside the bidet main body, a user function step in which the nozzle protrudes outside the bidet main body and sprays cleansing water to the user based on the user's operation, a sterilization preparation step in which a state where the nozzle protrudes outside the bidet main body is maintained to drain water from around the nozzle, and a sterilization step in which the nozzle is sterilized by ultraviolet (UV) light, and
wherein, in the sterilization preparation step, a state in which the nozzle protrudes more outward than a position of the nozzle in the sterilization step is maintained for a predetermined time.

12. The bidet of claim 11, further comprising a nozzle housing that provides a space in which the nozzle is accommodated,
wherein the nozzle and the nozzle housing are configured to prevent ultraviolet light from being emitted to the outside of the nozzle housing when the sterilization step is performed.

13. The bidet of claim 11, wherein, in the sterilization preparation step, a state in which the nozzle protrudes more outward than a position of the nozzle in the standby step is maintained for a predetermined time.

14. The bidet of claim 11, further comprising an operating unit that includes a plurality of operating buttons operated by the user to activate at least one of the bidet main body and the nozzle,
wherein the plurality of operating buttons include:
a cleansing operation button operated by the user to cause the user function step to be performed; and
a sterilization operation button operated by a user to cause the sterilization step to be performed,
wherein the sterilization step is performed when the sterilization operation button is operated by the user.

15. The bidet of claim 14, further comprising:
a toilet seat rotatably mounted to the bidet main body and configured for a user to be seated on; and
a seat sensor that detects whether the user is seated on the toilet seat,
wherein when the sterilization step is not performed for a preset sterilization standby time after a previous sterilization step was performed, the controller performs the sterilization step.

16. The bidet of claim 15, wherein the controller controls the nozzle to further perform a cleansing preparation step in which the nozzle is cleaned when the seat sensor detects that a user is seated, and a cleaning step in which the nozzle is cleaned.

17. The bidet of claim 16, wherein an extent to which the nozzle protrudes outward in the sterilization preparation step is smaller than an extent to which the nozzle protrudes outward in the user function step, and is the same as an extent to which the nozzle protrudes outward in the cleansing preparation step.

18. The bidet of claim 16, wherein a position where the nozzle is disposed in the sterilization preparation step is closer to a position where the nozzle is disposed in the standby step than to a position where the nozzle is disposed in the user function step.

19. The bidet of claim 16, wherein the sterilization standby time is longer than a time for which the cleansing preparation step is performed, and
a time for which the sterilization preparation step is performed is shorter than the sterilization standby time.

20. The bidet of claim 16, wherein an amount of water on an outer surface of the nozzle after the sterilization preparation step is completed is less than at least one of an amount of water on the outer surface of the nozzle after the cleansing preparation step is completed, an amount of water on the outer surface of the nozzle after the user function step is completed, and an amount of water on the outer surface of the nozzle after the cleaning step is completed.
